(19) United States
(12) Patent Application Publication
(10) Pub. No.: US 2026/0199011 A1
(43) Pub. Date: Jul. 16, 2026

(54) LIVE DATA INDICATOR BY OPTICAL PATH

(71) Applicant: Lumenis Ltd., Yokneam Ilit (IL)

(72) Inventor: Youssef Laham, Yokneam Ilit (IL)

(73) Assignee: Lumenis Ltd., Yokneam Ilit (IL)

(21) Appl. No.: 19/448,623

(22) Filed: Jan. 14, 2026

Related U.S. Application Data
(60) Provisional application No. 63/745,214, filed on Jan. 14, 2025.

(51) Int. Cl. A61B 18/22; A61B 90/00; A61B 18/00

(52) U.S. Cl. CPC A61B 18/22; A61B 90/37; A61B 2018/00982; A61B 2018/2255

(57) ABSTRACT

The present disclosure provides a medical laser console device and method utilizing an additional visible laser beam as a live data indicator. The indicator beam, visible in the endoscope's camera field of view, conveys real-time procedural information by manipulating its characteristics such as color, shape, or temporal pattern. This approach keeps the physician's focus on the target site, potentially improving procedural outcomes and reducing fatigue. The system can use existing hardware or software to create the indicator beam, simplifying visual information presentation.

### CROSS-REFERENCE TO RELATED APPLICATIONS

This application claims the benefit of U.S. Provisional Patent Application Serial No. 63/745,214 filed January 14, 2025, the disclosure of which is incorporated herein by reference.

### TECHNICAL FIELD

The present disclosure generally relates to surgical laser system. Particularly, but not exclusively, the present disclosure relates to surgical laser systems used in endoscopies.

### BACKGROUND

Medical lasers are used in a variety of procedures. Among several the procedures, laser energy is directed towards a target using a fiber as a conduit for the therapeutic laser energy. An endoscopic probe, with a camera or other sensor, is inserted into the patient to perform such procedures. A medical laser console is utilized to generate the necessary laser energy.

During a procedure, the operating room typically contains various displays to inform the clinicians, physicians, and other assisting parties of data relevant to the current procedure. Additionally, historical data, procedural parameters, and data generated by artificial intelligence introduce more actionable data into the operating room, spread onto several displays. A need therefore exists for a device and method to present the data in a more condensed manner.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The present disclosure provides a medical laser console device and method that utilizes an additional visible laser beam as a live data indicator. The medical laser console device generates the indicator laser beam, which is then directed to the end of an optical fiber, along with a therapeutic laser beam. The endoscope includes a camera such that the therapeutic beam and the indicator laser beam are simultaneously visible in the camera’s field of view.

In some embodiments, the medical laser console comprises a processor and optical system configured to produce a therapeutic laser beam and at least one additional light beam. The processor may receive an indication of a parameter of a medical procedure. In response, the

processor of the medical laser console will generate a control signal causing the optical system to generate both the therapeutic laser beam and an indicator laser beam using one of the additional light beams. The beams are then optically coupled to the proximal end of the optical fiber inserted into a working channel of an endoscope such that the indicator laser beam is visible in the field of view of the endoscope’s camera.

In further embodiments, the therapeutic beam is in the field of view of the endoscope coincident to the indicator laser beam.

In further embodiments, the field of view of the endoscope is displayed on an external display device.

In further embodiments, the field of the endoscope is displayed on an external display device along with procedural data or procedural parameters.

In further embodiments, the method can comprise sending a control signal to the laser source to cause the laser source to generate the laser light having one or more characteristics, characteristics comprising color, shape, intensity, or temporal pattern of the laser beam.

In further embodiments, the optical system manipulates the indicator laser beam by changing at least one characteristic of the indicator laser beam to convey information to the user. Characteristics comprising color, shape, intensity, or temporal pattern of the laser beam.

In further embodiments, the control signal defines the characteristics of the therapeutic laser beam and the additional laser beam.

In further embodiments, the light beam used to generate the indicator laser beam is a laser beam.

In further embodiments, the light beam used to generate the indicator laser beam is an aiming beam.

In further embodiments, the light beam used to generate the indicator laser beam originates from the same lasing source as the therapeutic laser beam.

In further embodiments, the light beam used to generate the indicator laser beam originates from a different lasing source as the therapeutic laser beam.

In further embodiments, the light beam used to generate the indicator laser beam originates from an infrared diode.

In further embodiments, the light beam used to generate the indicator laser beam originates from a color-changing LED.

In further embodiments, the light beam used to generate the indicator laser beam originates from a laser source laser source comprising either a Holmium based lasing medium or a Thulium based lasing medium.

Some embodiments can be implemented to provide a computer implemented method. The computer implemented method can comprise receiving an indication of a parameter of a medical procedure, wherein the medical laser console comprises an optical system configured to generate a therapeutic laser beam and at least one additional light beam; generating a control signal for the optical system, the control signal causing the optical system to generate an indicator laser beam using one of the at least one additional light beams.

In further embodiments, the optical system of the medical laser console is configured to couple to an optical fiber such that the therapeutic laser beam and the indicator laser beam are optically coupled to a proximal end of the optical fiber.

In further embodiments of the computer implemented method, the optical fiber is arranged to be inserted through a working channel of an endoscope.

In further embodiments, the indicator laser beam is in the field of view of the endoscope.

In further embodiments, the indicator laser beam is in the field of view of the endoscope coincident to the indicator laser beam.

In further embodiments, the optical system manipulates the indicator laser beam by changing at least one characteristic of the indicator laser beam to convey information to the user, wherein characteristics comprise color, shape, intensity, or temporal pattern.

In further embodiments, the control signal defines the characteristics of the therapeutic laser beam and the additional light beam. The characteristics comprising frequency, color, pulse duration, power output, and power density of each beam.

In further embodiments, the computer implemented method can comprise executing, by the processor, a machine learning (ML) model to store preferred settings, be trained to identify frames affected by laser interference, optimize utilization procedure parameters, provide real-time feedback, and the like.

Some embodiments can be implemented to provide an apparatus for a medical laser console device. The apparatus can comprise a processor; and memory coupled to the processor, the memory comprising instructions, which when executed by the processor cause the processor to implement the method of any of the embodiments described herein.

Some embodiments can be implemented as a computer-readable storage device comprising instruction, which when executed by a processor of a medical laser system cause the medical laser console to implement the method of any of the embodiments described herein.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1A illustrates an example endoscopic procedure system, in accordance with at least one embodiment.

FIG. 1B illustrates a portion of the endoscopic procedure system of FIG. 1A in greater detail.

FIG. 2 illustrates the field of view of an endoscopic camera.

FIG. 3 is a block method diagram in accordance with at least one embodiment.

FIG. 4 illustrates computing environment, in accordance with at least one embodiment.

FIG. 5 illustrates a computer-readable storage medium in accordance with at least one embodiment.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

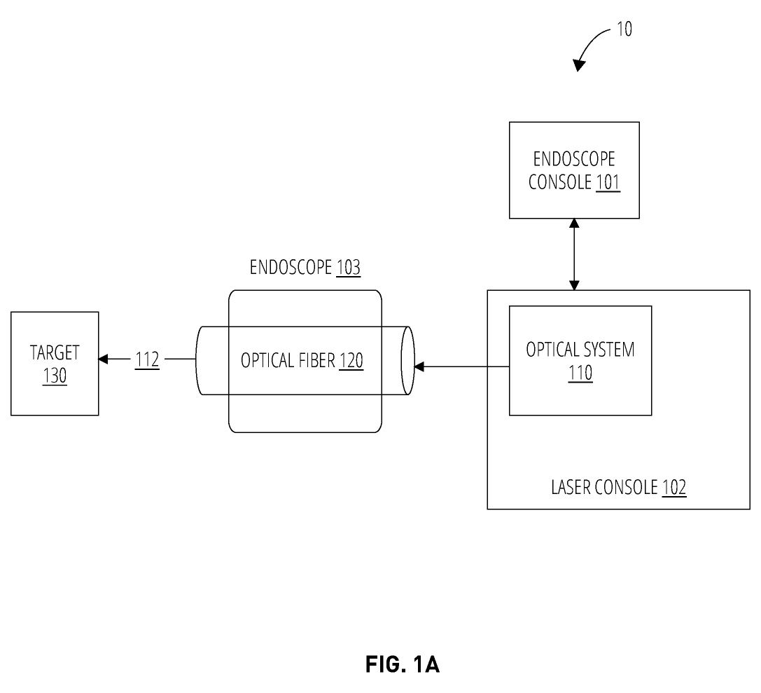

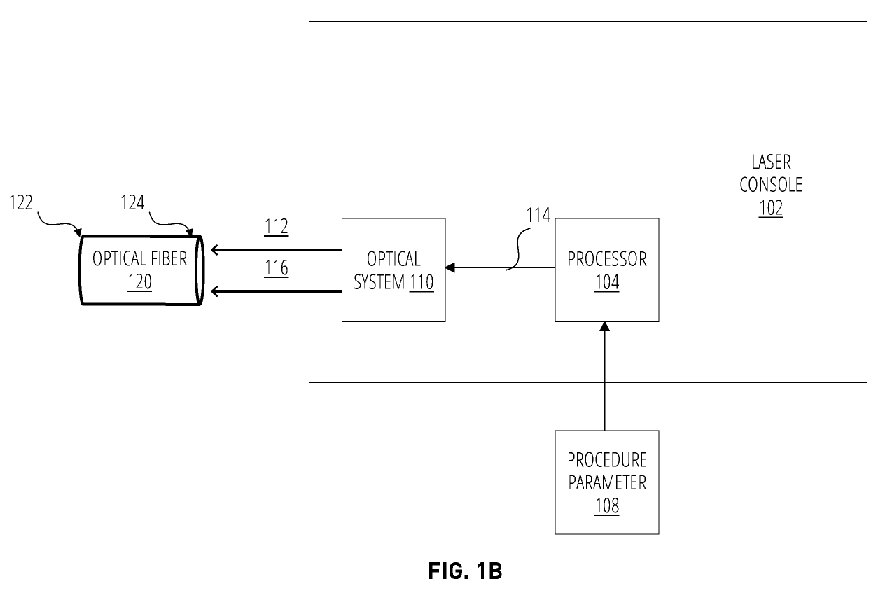

FIG. 1A shows an exemplary endoscopic procedure system 10, in accordance with some embodiments of the present disclosure. In some embodiments, the exemplary endoscopic procedure system 10 comprises a laser console 102 and an optical fiber 120 configured to emit a therapeutic laser beam 112 towards a target 130 during operation. In some embodiments, the therapeutic laser beam 112 can be a pulsed laser beam. The therapeutic laser beam 112 can be directed to target 130 by positioning the distal end of the therapeutic laser beam 112 proximate to the target 130. For example, in some embodiments, target 130 can be a tissue, a stone, a tumor, a cyst, and the like located in a lumen or cavity in a patient’s body. As a specific example, target 130 can be a urinary stone located in a kidney or other urinary track, lumen, or cavity. Often, target 130 can be free floating in a liquid medium (e.g., urine, water, etc.) The therapeutic laser beam 112 can be inserted into the patient’s cavity via a working channel of a ureteroscope (not shown) and positioned such that the distal end of the therapeutic laser beam 112 extends out of the working channel and is proximate to the target 130. The target 130 can be exposed to the therapeutic laser beam 112 to treat (e.g., ablate, disintegrate, dust, shatter, or the like) the target 130.

The desired nature and character of therapeutic laser beam 112 are dictated by the parameters of the procedure. Procedure parameters may include the laser pulse characteristics of the therapeutic laser beam 112, such as, but not limited to, the laser’s frequency, color, pulse duration, power output, and power density. Procedure parameters may also include data indicative of the target’s character (e.g., tissue type or stone composition). Procedure parameters may also include endoscope settings or configurations. Procedure parameters may also include patient-specific information relevant to the procedure. Procedure parameters may also include live feedback data from the endoscopic system or feedback data from other relevant equipment in operation.

In some embodiments, a camera may be included in the endoscope 103 to visualize the procedure and capture the internal environment during medical procedures. In some embodiments, the camera feed may be displayed live on the endoscope console 101 or an external display device for physician’s reference during the procedure. The camera feed may also be recorded for later viewing and assessment.

Turning to FIG. 1B, the medical laser console 102 can include a processor 104 and an optical system 110.

Processor 104 may be configured to send and receive control signals and/or information elements, such as the procedure parameters 108, as well as store computer-readable memory storing instructions for later generation of laser or light beams. Additionally, processor 104 may be configured to send information elements, including procedure parameters 108, characteristics of the therapeutic laser beam 112, patient data, and procedural data to an external display device (not shown).

Optical system 110 may include at least one laser source (not illustrated) or at least one light source (not illustrated) to generate a laser beam or a light beam. Laser sources may include a laser medium and a pump light source configured to generate a laser beam. The laser source can include any of a variety of laser mediums and pump light sources. For example, laser source can be a Holmium (Ho) based laser (e.g., Ho:YAG, or the like), a Chromium (Cr), Thulium (Tm), and Ho based laser (e.g., CTH:YAG, or the like). As another example, the laser source can be a Thulium (Tm) based fiber laser (TFL). The pump light sources can be capacitor driven flash lamps, light emitting diodes (LEDs), or the like. The generated laser beams may take several forms, including but not limited to solid-state lasers, gas lasers, diode lasers, and fiber lasers. In other instances, the optical system 110 may generate a light beam using light sources such as capacitor driven flash lamps, light emitting diodes (LEDs), or the like. Other optical elements may also be included in optical system 110, including, but not limited to lenses, polarizers, beam splitters, beam combiners, light detectors, wavelength division multiplexers, collimators, circulators, etc.

Upon the direction of processor 104, optical system 110 is configured to generate at least the therapeutic laser beam 112 and at least one additional light beam. The additional light beam may comprise another laser beam, an aiming beam (a beam designed to illuminate or highlight the target), or a light emitted from an infrared (IR) diode or LED. Processor 104 may then generate a control signal 114 for the optical system to generate an indicator laser beam 116 from one of the additional light beams. It is important to note that the indicator laser beam 116 will be in the visible spectrum. As such, often the additional light beam may be in the visible spectrum. However, in some examples, the additional light beam may be non-visible (e.g. infrared) while the optical system may convert the non-visible light to visible light (e.g., via frequency-doubling, or the like). In the embodiments where only one additional light beam is generated and the additional light beam is a laser beam with the appropriate characteristics, the change to status as an indicator laser beam may be merely semantic. However, in the instances wherein a plurality of additional light beams was initially generated, any of the additional light beams may be used to generate the indicator laser beam 116, even if a light beam with the appropriate characteristics exists. The efficiencies and methods of selecting which among additional light beams to use in generating the indicator light beam is understood to be a separate matter of preference from the present invention, as any of the additional light beams may be used in the claimed methods and devices.

The indicator laser beam 116 serves as a data indicator, conveying actionable information to the physician in real-time. As subsequent procedure parameters 108 or procedural data are fed into the processor 104 of laser console 102, the characteristics of the indicator laser beam 116 may be manipulated. The manipulation of the characteristics of indicator laser beam 116, such as color, shape, or flicker frequency, can communicate various types of data live and directly within the physician's field of view, eliminating the need to look away from the target site. This capability keeps the physician's eyes and cognitive focus on the task at hand, potentially reducing fatigue and improving overall procedural outcomes. Additionally, because the indicator laser beam is displayed in the endoscopic camera feed, use of the indicator laser beam reduces dependency on external hardware or software for data display, resulting in a more streamlined procedural environment.

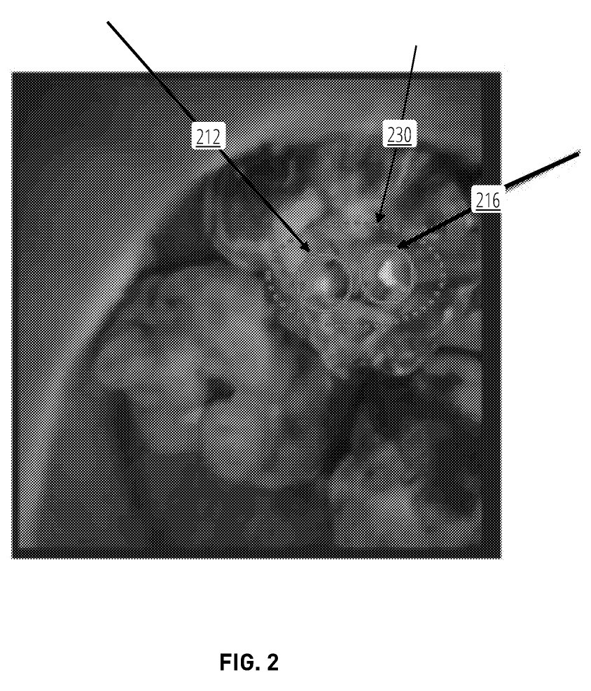

As illustrated in FIG. 2, the indicator laser beam 216 may be generated from an aiming beam. In those embodiments, the indicator laser beam 216 may also continue serving the purposes of the aiming beam, highlighting and illuminating the target area 230 for the medical procedure, while also functioning as a live data indicator. This dual functionality allows the physician to receive visual guidance and data within the camera’s field of view using a single beam. By combining these functions, the system simplifies the visual information presented on the camera's display, reducing visual clutter and improving readability with minimal impact to user flow.

Returning to FIG. 1B, after the optical system has generated the therapeutic laser beam 112 and the indicator laser beam 116 are then directed to the target site via optical fiber 120. Optical fiber 120 comprises a proximal end 124 and a distal end 122. The proximal end 124 is the end of the optical fiber 120 coupled to the laser console 102 and through which light beams enter while the distal end 122 is the end of the optical fiber 120 through which light beams are emitted and via which light beams can be directed onto the target site.

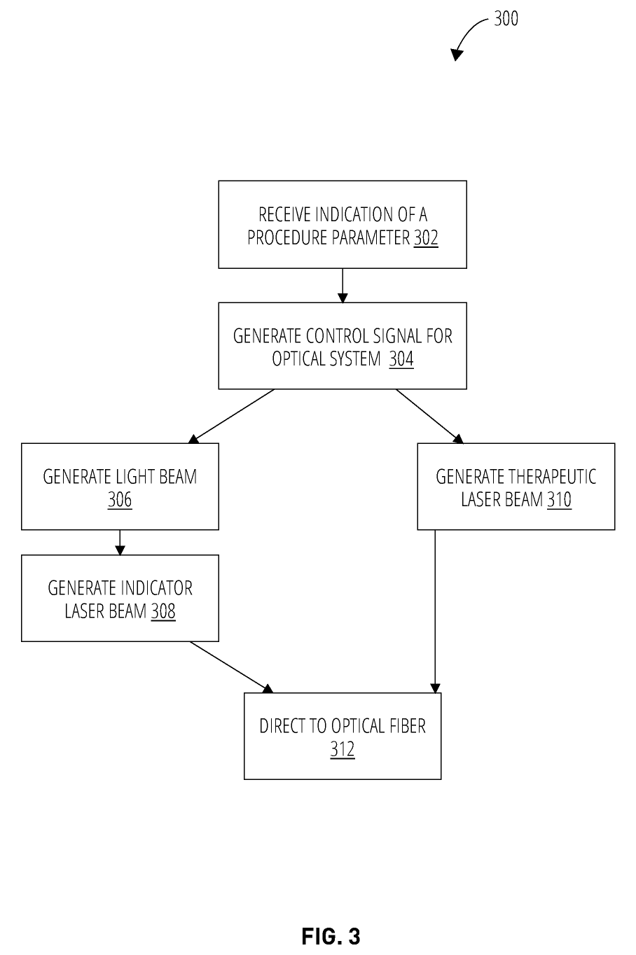

FIG. 3 illustrates a method 300 block diagram of the method in which the indicator laser beam is generated. Method 300 can be implemented by the medical laser console.

Method block 302 represents the medical laser console receiving an indication of a procedure parameter or change of procedure parameter. Upon receipt, the processor of the medical laser console generates a control signal for the optical system, as shown in method block 304.

Method blocks 306 and 310 occur upon receipt of the control signal by the optical system. Upon receipt of the control signal, the optical system then generates the therapeutic laser beam (method block 310) and at least one additional light beam (method block 306). The generated light beam may be sourced from a laser source or a light source or combination of the two. In some embodiments, the source of the light beam is external to the hardware of the medical laser console, but the generation of the light beam is prompted by the software commands of the medical laser console, nonetheless.

Method block 308 represents the generation of the indicator laser beam. At least one of the generated light beams are used to further generate the indicator laser beam. The indicator laser beam differs in purpose in comparison to the preceding light beam, in that it is configured to be manipulated upon the subsequent input of procedural data, patient data, procedural parameters, equipment feedback, and the like. However, in some embodiments, the indicator laser beam may retain the purposes of the preceding light beam. For example, the indicator laser beam may have been generated from an aiming beam; in that example, the indicator laser beam may continue to serve the purpose of the aiming beam in highlighting and illuminating the target area.

Method block 312 represents the emission of the indicator laser beam and the therapeutic laser beam to the optical fiber of the endoscope. In some embodiments, additional light or laser beams may be directed to proximal end of the optical fiber along with the indicator laser beam and the therapeutic laser beam. Additional light or laser beams may be useful for the other needs of the procedure, further illumination, calibration or alignment, console settings, establishment of feedback mechanisms, availability of multiple treatment modalities, diagnoses, safety features, patient-specific customization, and/or physician preferences, among other reasons.

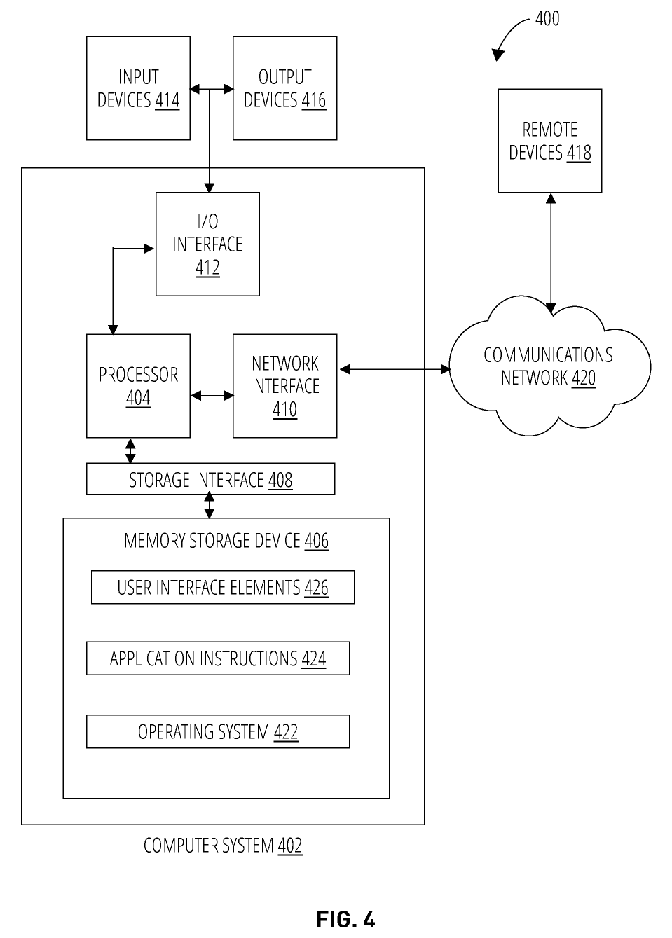

FIG. 4 is a block diagram of a computing environment 400 including a computer system 402 for implementing embodiments consistent with the present disclosure. In some embodiments, the computing environment 400, or portion thereof (e.g., the computer system 402) may comprise or be comprised in a laser system. Accordingly, in various embodiments, computer system 402 may determine a power of laser light emitted from a distal end of an optical fiber based on a duration of existence of a bubble formed by the laser light.

The computer system 402 may include a central processing unit (“CPU” or “processor”) 404. The processor 404 may include at least one data processor for executing instructions and/or program components for executing user or system-generated processes. A user may include a person, a person using a device such as those included in this disclosure, or another device. The processor 404 may include specialized processing units such as integrated system (bus) controllers, memory management control units, floating point units, graphics processing units, neural processing units, digital signal processing units, etc. The processor 404 may be disposed in communication with input devices 414 and output devices 416 via I/O interface 412. The I/O interface 412 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n /b/g/n/x, Bluetooth, cellular (e.g., Code-Division Multiple Access (CDMA), High-Speed Packet Access (HSPA+), Global System For Mobile Communications (GSM), Long-Term Evolution (LTE), WiMAX, or the like), etc.

Using the I/O interface 412, computer system 402 may communicate with input devices 414 and output devices 416. In some embodiments, the processor 404 may be disposed in communication with a communications network 420 via a network interface 410. In various embodiments, the communications network 420 may be utilized to communicate with a remote memory storage device 406, such as for accessing look-up tables, performing updates, or utilizing external resources. The network interface 410 may communicate with the communications network 420. The network interface 410 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc.

The communications network 420 can be implemented as one of the different types of networks, such as intranet or Local Area Network (LAN), Closed Area Network (CAN) and such. The communications network 420 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 420 may include a variety of network devices, including routers, bridges, servers, computing devices, storage devices, etcetera. In some embodiments, the processor 404 may be disposed in communication with a memory storage device 406 via a storage interface 408. The storage interface 408 may connect to memory storage device 406 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etcetera.

Furthermore, memory storage device 406 may include one or more computer-readable storage media utilized in implementing embodiments consistent with the present disclosure. Generally, a computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media.

The memory storage device 406 may store a collection of program or database components, including, without limitation, an operating system 422, an application instructions 424, and a user interface elements 426. In various embodiments, the operating system 422 may facilitate resource management and operation of the computer system 402. Examples of operating systems include, without limitation, APPLE^{®} MACINTOSH^{®} OS X^{®}, UNIX^{®}, UNIX-like system distributions (E.G., BERKELEY SOFTWARE DISTRIBUTION^{®} (BSD), FREEBSD^{®}, NETBSD^{®}, OPENBSD, etc.), LINUX^{®} DISTRIBUTIONS (E.G., RED HAT^{®}, UBUNTU^{®}, KUBUNTU^{®}, etc.), IBM^{®}OS/2^{®}, MICROSOFT^{®} WINDOWS^{®} (XP^{®}, VISTA^{®}/7/8, 10 etc.), APPLE^{®} IOS^{®}, GOOGLE^{TM} ANDROID^{TM}, BLACKBERRY^{®} OS, or the like.

The application instructions 424 may include instructions that when executed by the processor 404 cause the processor 404 to perform one or more techniques, steps, procedures, and/or methods described herein.

The user interface elements 426 may facilitate display, execution, interaction, manipulation, or operation of program components through textual or graphical facilities. For example, user interfaces may provide computer interaction interface elements on a display system operatively connected to the computer system 402, such as cursors, icons, checkboxes, menus, scrollers, windows, widgets, etcetera. The user interface elements 426 may be employed by application instructions 424 and/or operating system 422 to provide, for example, a user interface with which a user can interact with computer system 402. In some embodiments, the user interface elements 426 may be displayed on a display.

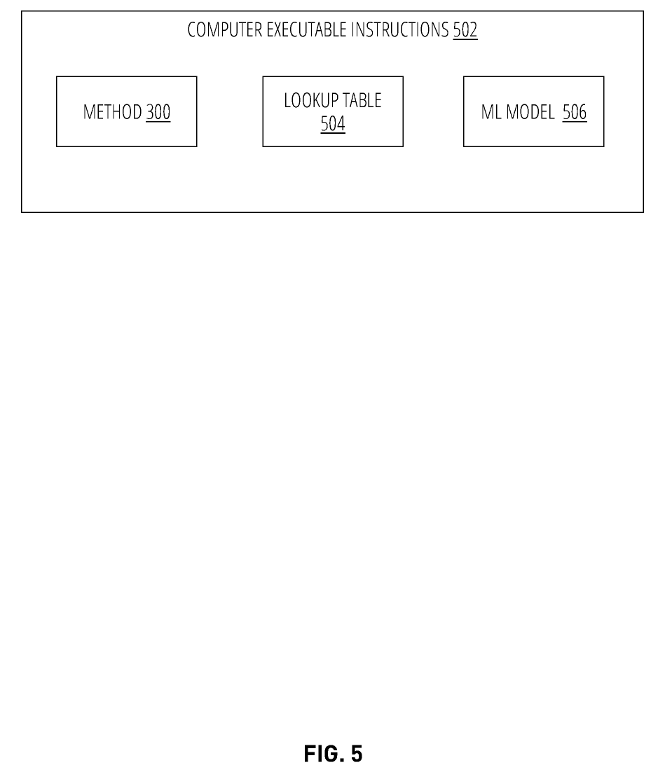

FIG. 5 illustrates computer-readable storage medium 500. Computer-readable storage medium 500 may comprise any non-transitory computer-readable storage medium or machine-readable storage medium, such as an optical, magnetic or semiconductor storage medium. In various embodiments, computer-readable storage medium 500 may comprise an article of manufacture. In some embodiments, 500 may store computer executable instructions 502 with which circuitry (e.g., processor 404, or the like) can execute. For example, computer executable instructions 502 can include instructions to implement operations described with respect to method 500, computer executable instructions lookup table 504, or ML Model 506. Examples of computer-readable storage medium 500 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 502 may include any suitable type of code, such as source code, compiled code, interpreted code, executable code, static code, dynamic code, object-oriented code, visual code, and the like.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A medical laser console, comprising:
a processor; and
memory coupled to the processor, the memory comprising instructions, which when executed by the processor cause the processor to:
receive an indication of a parameter of a medical procedure, wherein the medical laser console comprises an optical system configured to generate a therapeutic laser beam and at least one additional light beam;
generate a control signal for the optical system, the control signal to cause the optical system to generate an indicator laser beam using one of the at least one additional light beam,
wherein the optical system of the medical laser console is configured to couple to an optical fiber such that the therapeutic laser beam and the indicator laser beam are optically coupled to a proximal end of the optical fiber,
wherein the optical fiber is configured to be inserted into a working channel of an endoscope.

2. The medical laser console of claim 1, wherein the therapeutic beam is in the field of view of the endoscope coincident to the indicator laser beam.

3. The medical laser console of claim 2, wherein the instructions, when executed, further cause the processor to display the field of view of the endoscope on an external display device.

4. The medical laser console of claim 3, wherein the instructions, when executed, further cause the processor to display at least one of procedural data or the parameter of the medical procedure on the external display device.

5. The medical laser console of claim 4, wherein the procedural data includes data generated by an artificial intelligence system analyzing the medical procedure.

6. The medical laser console of claim 1, wherein the optical system manipulates the indicator laser beam by changing at least one characteristic of the indicator laser beam to convey information to a user, wherein the at least one characteristic comprises wavelength, shape, intensity, or temporal pattern.

7. The medical laser console of claim 1, wherein the at least one light beam comprises an aiming beam.

8. The medical laser console of claim 7, wherein the medical laser console comprises at least one therapeutic laser source configured to generate the therapeutic laser beam and at least one additional laser source configured to generate the at least one light beam.

9. The medical laser console of claim 8, wherein the at least one additional laser source comprises a color-changing LED.

10. A computer-readable storage device comprising instruction, which when executed by a processor of a medical laser console causes the medical laser console to:
receive an indication of a parameter of a medical procedure, wherein the medical laser console comprises an optical system configured to generate a therapeutic laser beam and at least one light beam;
generate a control signal for the optical system, the control signal to cause the optical system to generate an indicator laser beam using one of the at least one light beams,
wherein the optical system of the medical laser console is configured to couple to an optical fiber such that the therapeutic laser beam and the indicator laser beam are optically coupled to a proximal end of the optical fiber,
wherein the optical fiber is configured to be inserted into a working channel of an endoscope, and
wherein the indicator laser beam is in a field of view of the endoscope.

11. The computer-readable storage device of claim 10, wherein the optical system manipulates the indicator laser beam by changing at least one characteristic of the indicator laser beam to convey information to a user, wherein the at least one characteristic comprises color, shape, intensity, or temporal pattern.

12. The computer-readable storage device of claim 10, wherein the control signal defines characteristics of the therapeutic laser beam and the indicator laser beam, the characteristics comprising frequency, wavelength, pulse duration, power output, and power density of each beam.

13. The computer-readable storage device of claim 10, wherein the therapeutic beam is in the field of view of the endoscope coincident to the indicator laser beam.

14. The computer-readable storage device of claim 13, wherein the instructions, when executed, further cause the medical laser console to display the field of view of the endoscope on a display device.

15. The computer-readable storage device of claim 14, wherein the instructions, when executed, further cause the medical laser console to display at least one of procedural data or the parameter of the medical procedure on the external display device.

16. A method for a medical laser console, comprising:
receiving, at a processor of the medical laser console, an indication of a parameter of a medical procedure, wherein the medical laser console comprises an optical system configured to generate a therapeutic laser beam and at least one light beam; and
generating, at the processor, a control signal for the optical system, the control signal to cause the optical system to generate an indicator laser beam using one of the at least one light beams,
wherein the optical system of the medical laser console is configured to couple to an optical fiber such that the therapeutic laser beam and at the indicator laser beam are optically coupled to a proximal end of the optical fiber,
wherein the optical fiber is configured to be inserted into a working channel of an endoscope, and
wherein the indicator laser beam is visible in a field of view of an endoscope.

17. The method of claim 16, wherein the therapeutic beam is in the field of view of the endoscope coincident to the indicator laser beam.

18. The method of claim 16, further comprising displaying the field of view of the endoscope on an external display device.

19. The method of claim 18, further comprising displaying at least one of procedural data or the parameter of the medical procedure on the external display device, wherein the procedural data includes data generated by an artificial intelligence system analyzing the medical procedure.

20. The method of claim 16, wherein the optical system manipulates the indicator laser beam by changing at least one characteristic of the indicator laser beam to convey information to a user, wherein the at least one characteristic comprises color, shape, intensity, or temporal pattern.
